(19) United States
(12) Patent Application Publication
(10) Pub. No.: US 2025/0146082 A1
(43) Pub. Date: May 8, 2025

(54) Methods for Enriching Microbial Cell-Free DNA in Plasma

(71) Applicant: Wisconsin Alumni Research Foundation, Madison, WI (US)

(72) Inventors: Mehreen Kisat, Middleton, WI (US); Bradon McDonald, Madison, WI (US); Eddie Dominguez, Madison, WI (US)

(73) Assignee: Wisconsin Alumni Research Foundation, Madison, WI (US)

(21) Appl. No.: 18/936,679

(22) Filed: Nov. 4, 2024

Related U.S. Application Data
(60) Provisional application No. 63/596,705, filed on Nov. 7, 2023.

(51) Int. Cl. C12Q 1/6886; C12N 15/10; C12Q 1/6806; C12Q 1/6855; C12Q 1/6869; C12Q 1/6888

(52) U.S. Cl. CPC C12Q 1/6886; C12N 15/1093; C12Q 1/6806; C12Q 1/6855; C12Q 1/6869; C12Q 1/6888; C12Q 2600/106; C12Q 2600/156

(57) ABSTRACT

Described herein is a method of enriching a body fluid sample from a host subject for a ratio of non-native cell-free DNA (e.g., microbial cell-free DNA) to host subject cell-free DNA (host cfDNA). The method can include obtaining the body fluid sample from the host subject; extracting total cell-free DNA (total cfDNA) from the body fluid sample; preparing a single-stranded DNA library from the total cfDNA, wherein the single-stranded DNA library is enriched for cfDNA existing in a single-stranded configuration; and selecting a subset of the single-stranded DNA library based on the fragment size to provide a size-selected cfDNA library, wherein the size-selected cfDNA library has a DNA fragment length of 110 nucleotides or less and is enriched for the ratio of non-native cell-free DNA to host cfDNA.

### CROSS-REFERENCE TO RELATED APPLICATIONS

This application claims priority to U.S. Provisional Application 63/596,705 filed on Nov. 7, 2023, which is incorporated herein by reference in its entirety.

### STATEMENT REGARDING FEDERALLY SPONSORED RESEARCH & DEVELOPMENT

This invention was made with government support under GM148858 awarded by the National Institutes of Health. The government has certain rights in the invention.

### FIELD OF THE DISCLOSURE

The present disclosure is related to methods for enriching samples for microbial cell-free DNA and other small non-native DNAs.

### BACKGROUND

Rapid identification of sepsis and the causative pathogen(s) in a specific patient is critical to patient management. Also, in patients with a traumatic injury, the inflammatory response due to the trauma complicates the clinical picture and can obscure the development of infection. It can be difficult to differentiate inflammatory response related to the initial tissue injury from sepsis. While microbial cultures are the gold standard in diagnosing infections, microbial cultures are hampered by low sensitivity, lack of quantitative resolution, and results which can take three days or longer to obtain. Patients are often immediately started on broad spectrum antibiotics. This leads to the growing challenge of antimicrobial resistance in hospitals and in the community. Earlier identification of sepsis and pathogen(s) causing sepsis can lead to more effective antimicrobial treatment.

Plasma metagenomic sequencing has shown promise as a rapid diagnostic tool for sepsis. Microbial cell-free DNA (cfDNA) can be detected in blood, particularly in patients with ongoing infections. However, microbial cfDNA is highly degraded and contributes a very small fraction of DNA in plasma. It has previously been shown that microbial cfDNA fragments are shorter in plasma compared to human cfDNA (cfDNA) fragments. See, Kisat et al., “Plasma metagenomic sequencing to detect and quantify bacterial DNA in ICU patients suspected of sepsis: A proof of principle study”, J Trauma Acute Care Surg, 91:6, pp. 988-994 (2021). This observation was previously used to enrich the microbial cfDNA signal in plasma metagenomic sequencing by size selection of sequencing libraries. Specifically, the contribution of longer human cfDNA fragments was reduced by selecting them out (U.S. Publication No. 2019/0153512).

What is needed are further refinements to allow for additional enrichment of microbial cfDNA in plasma samples, particularly samples from patients suspected of infection with a microbial pathogen.

### BRIEF SUMMARY

In an aspect, a method of enriching a body fluid sample from a host subject for ratio of non-native cell-free DNA (cfDNA) to host subject cfDNA comprises obtaining the body fluid sample from the host subject; extracting total cfDNA from the body fluid sample; preparing a single-stranded DNA library from the total cfDNA, wherein the single-stranded DNA library is enriched for cfDNA existing in a single-stranded configuration; and selecting a subset of the single-stranded DNA library based on the fragment size to provide a size-selected cfDNA library, wherein the size-selected cfDNA library has a DNA fragment length of 110 nucleotides or less and is enriched for the ratio of non-native cfDNA to host cfDNA.

In another aspect, a method of enriching a body fluid sample from a host subject for a ratio of microbial cell-free DNA (mDNA) to host subject cell-free DNA (host cfDNA) comprises obtaining the body fluid sample from the host subject; extracting total cell-free DNA (total cfDNA) from the body fluid sample; preparing a single-stranded DNA library from the total cfDNA, wherein the single-stranded DNA library is enriched for cfDNA existing in a single-stranded configuration; and selecting a subset of the single-stranded DNA library based on fragment size to provide size-selected cfDNA library, wherein the size-selected cfDNA library has a DNA fragment length of 110 nucleotides or less and is enriched for the ratio of mDNA to host cfDNA.

In another aspect, a method of enriching a plasma sample from a host subject for ratio of circulating tumor DNA (ctDNA) to host subject cfDNA comprises obtaining the plasma sample from the host subject; extracting total cfDNA from the plasma sample; preparing a single-stranded DNA library from the total cfDNA, wherein the single-stranded DNA library is enriched for cfDNA existing in a single-stranded configuration; and selecting a subset of the single-stranded DNA library based on the fragment size to provide a size-selected cfDNA library, wherein the size-selected cfDNA library has a DNA fragment length of 110 nucleotides or less and is enriched for the ratio of ctDNA to host cfDNA.

### BRIEF DESCRIPTION OF THE DRAWINGS

FIGS. 1A and B. show the workflow for improving the recovery of microbial cell-free DNA using different library preparation protocols and size-selection. (1A), Plasma samples were processed using semi-automated magnetic bead-based extraction methods, followed by dsDNA and ssDNA library protocols, size-selection to exclude DNA fragments greater than 110 bp in length, and sequencing. (1B), Quantification of pooled libraries before and after application of size-selection for dsDNA and ssDNA library protocols.

FIG. 2 shows a comparison of microbial DNA fraction between paired samples (n=46) using double-stranded DNA (ds-DNA) and single-stranded DNA (ss-DNA) library preparation before and after size-selection (23-fold, p<0.0001 for dsDNA size-selected compared to dsDNA and 6-fold, p<0.0001 for ssDNA size-selected compared to ssDNA).

FIG. 3 shows a comparison of microbial fraction and cell-free DNA extraction yield in 1 ml of plasma across the four library preparation approaches.

FIG. 4 shows longitudinal patient-specific pathogen DNA quantification using the dsDNA size selected library preparation. Filled circles indicate detection of pathogen DNA significantly above background (see methods). Grey indicates non-significant pathogen DNA detection, white indicates no detection. Boxes below each plot indicate microbiology cultures. Color ranges indicate antimicrobial treatment regimens.

FIG. 5 shows longitudinal patient-specific pathogen DNA quantification using the dsDNA, ssDNA and ssDNA-size selected library preparations. Filled circles indicate detection of pathogen DNA significantly above background (see methods). Grey indicates non-significant pathogen DNA detection, white indicates no detection.

The above-described and other features will be appreciated and understood by those skilled in the art from the following detailed description, drawings, and appended claims.

### DETAILED DESCRIPTION

Circulating human cfDNA in human plasma, for example, includes short extracellular DNA fragments (approximately 160 to 180 bases in length). cfDNA in human bodily fluids such as plasma can also include non-human (i.e., non-native) cfDNA for example from microbes in addition to a substantial proportion of human cfDNA. For example, a human plasma sample may contain, in addition to human cfDNA, cfDNA of one or more commensal bacteria as well as cfDNA from one or more infection-causing pathogens, such as a pathogenic bacteria, viruses, and fungi. Further, in patients with cancer, a variable fraction of cfDNA in plasma is contributed by cancer cells compared to non-cancerous cells. Patient's microbial cfDNA profiles can vary with tumor type and can enable early detection of cancer. Also, circulating tumor DNA (ctDNA), DNA released by tumors into the bloodstream, can carry tumor-specific somatic genetic alterations. Analysis of circulating cfDNA from plasma has several potential diagnostic applications in treatment of infections, transplants, and cancer medicine.

Sequencing cfDNA in plasma and other body fluids can rapidly identify pathogens by classifying non-human sequencing reads to microbes and potential pathogens. However, <0.001% of cfDNA in circulation originates from microbial DNA, making previous approaches for pathogen identification expensive and time-consuming. Described herein is a method of preparing a sequencing library using single stranded DNA library (increasing contribution of microbial cfDNA) followed by reducing the contribution of long human cfDNA fragments (e.g., human cfDNA). The two approaches are complementary as they can simultaneously exclude longer human cfDNA fragments (using size selection) while incorporating larger amounts of shorter mDNA fragments into the sequencing library (using ssDNA library preparation). As shown herein, using a combination of single-stranded DNA (ssDNA) sequencing library preparation and size selection (SS) can enrich mDNA. However, at the genus level, this combination also increases background noise which limits sensitivity for pathogen detection.

In an aspect, a method of enriching a body fluid sample from a host subject for a ratio of non-native cell-free DNA (cfDNA) to host subject cell-free DNA (host cfDNA) comprises obtaining the body fluid sample from the host subject; extracting total cell-free DNA (total cfDNA) from the body fluid sample; preparing a single-stranded DNA library from the total cfDNA, wherein the single-stranded DNA library is enriched for cfDNA existing in a single-stranded configuration; and selecting a subset of the single-stranded DNA library based on the fragment size to provide size-selected cfDNA, wherein the size-selected cfDNA has a DNA fragment length of 110 nucleotides or less and is enriched for the ratio of non-native cfDNA to host cfDNA.

As used herein, host cfDNA or native cfDNA is cfDNA released from native cells of the host, while non-native DNA is DNA released from a microbe, a cancerous cell, or donor cell, for example. In an aspect, the non-native DNA is microbial cell-free DNA. Microbes can include those associated infections in different sites of the body such as urinary, pulmonary, gastrointestinal infections or blood.

In an aspect, a method of enriching a body fluid sample from a host subject for a ratio of microbial cell-free DNA (mDNA) to host subject cell-free DNA (host cfDNA) comprises obtaining the body fluid sample from the host subject; extracting total cell-free DNA (total cfDNA) from the body fluid sample; preparing a single-stranded DNA library from the total cfDNA, wherein the single-stranded DNA library is enriched for cfDNA existing in a single-stranded configuration; and selecting a subset of the single-stranded DNA library based on the fragment size to provide size-selected cfDNA, wherein the size-selected cfDNA has a DNA fragment length of 110 nucleotides or less and is enriched for the ratio of mDNA to host cfDNA.

Exemplary patients are those with infections detectable in the blood, urine and lungs, for example. Exemplary body fluid samples include plasma samples, urine samples, sputum samples, bronchoalveolar lavage, stool samples, peritoneal fluid, pleural fluid, cerebrospinal fluid, synovial fluid, and interstitial fluid.

In an aspect, a blood sample can be obtained using a Streck cell-free DNA stabilizing tube, for example. Blood can be centrifuged for 10 minutes at 820 g to provide a plasma sample.

In the case of a human patient, blood samples can be obtained during hospital admission of the host subject, such as after ICU admission, typically prior to the diagnosis of sepsis (unless the patient comes in with sepsis at the time of admission), such as 1, 2, 3, 4, 5, 6, 7, 8, 9 or 10 days after ICU admission, preferably less than or equal to 10 days after ICU admission. Advantageously, the methods allow differentiation of the inflammatory response from infection.

In another aspect, blood samples can be obtained after organ transplant to distinguish post-transplant infection from rejection. In an aspect, the blood sample can be obtained for acute or chronic rejection after organ transplant.

In another aspect, blood samples can be obtained from a cancer patient to perform a microbiome analysis in cancer patients. Samples can be obtained prior to detection of cancer, from asymptomatic patients and for cancer detection, for example.

The term “extraction” as used herein refers to any method for separating or isolating the nucleic acids from a sample, more particularly from a biological sample, such as blood or plasma. Nucleic acids such as RNA or DNA may be released, for example, by cell lysis. In an aspect, lysis is accomplished using Proteinase K and a lysis buffer. Following the lysis step, various methods known in the art can be used to isolate the DNA such as columns and magnetic beads, for example. In an aspect, extraction of the cfDNA can include use of the QIAamp® Circulating Nucleic Acid kit (QIAGEN) or Magmax™ cell-free DNA kit (THERMOFISHER) which provides recovery and concentration of cfDNA fragments.

As used herein, the term “total cf” DNA does not necessarily mean 100% of the cfDNA in a plasma sample, but rather a representative mixture of native cfDNA and any non-native cfDNA in the sample. For a typical plasma sample, 1 ml of plasma typically yields 5-10 ng of cfDNA.

The method includes preparing a single-stranded DNA library from the total cfDNA wherein the single-stranded DNA library is enriched for the ratio of single-stranded DNA in an admixture of various forms of cfDNA. In an aspect, the single-stranded DNA library is produced using a Single Reaction Single-stranded LibrarY (SRSLY) approach (Troll et al. “A ligation-based single-stranded library preparation method to analyze cell-free DNA and synthetic oligos”, BMC Genomics, (2019) 20:1023). In this method, a phosphorylation step simultaneously prepares DNA molecules for ligation without end-polishing and simultaneously ligates proprietary Illumina®-compatible adapters. End-polishing is a method which provides blunt ends at the end of dsDNA molecules by filling-in 5′ overhangs and digesting 3′ overhangs. Thus, in an aspect, a single-stranded DNA library is prepared using a single step phosphorylation/ligation reaction in which next generation sequencing (NGS) adaptors are ligated to the size-selected DNA to provide the single-stranded DNA library.

In an aspect, prior to the phosphorylation/ligation dual reaction, the DNA is coated with single-stranded DNA binding protein.

In an aspect, in a first step, the cfDNA is heat denatured and cold shocked to produce single-stranded cfDNA. A thermostable single-stranded DNA binding protein (SSB) is added to maintain the single-stranded cfDNA by coating the single-stranded cfDNA with the SSB. The SSB coated single-stranded cfDNA is then reacted in a phosphorylation/ligation dual reaction with forward and reverse dsDNA NGS adaptors containing single-stranded overhangs. The single-stranded overhangs are adapted to maintain the native 5′-3′ polarity of the DNA molecules. In the phosphorylation/ligation dual reaction, T4 polynucleotide kinase phosphorylates 5′ termini and dephosphorylates 3′ termini to prepare the DNA termini for ligation of the NGS adaptors. T4 DNA ligase ligates the adaptors to the ssDNA, at which point the ssDNA library is complete and ready for sequencing.

The term “library,” as used herein refers to a plurality of genome/transcriptome-derived sequences. The library may also have sequences allowing amplification of the “library” by the polymerase chain reaction or other in vitro amplification methods well known to those skilled in the art. In various embodiments, the library may have sequences that are compatible with next-generation high throughput sequencing platforms. In some embodiments, as a part of the sample preparation process, “barcodes” may be associated with each sample. In this process, short oligonucleotides are added to primers, where each different sample uses a different oligo in addition to a primer.

In certain embodiments, primers and barcodes are ligated to each sample as part of the library generation process. Thus, during the amplification process associated with generating the amplicon library, the primer and the short oligo are also amplified. As the association of the barcode is done as part of the library preparation process, it is possible to use more than one library, and thus more than one sample. Synthetic nucleic acid barcodes may be included as part of the primer, where a different synthetic nucleic acid barcode may be used for each library. In some embodiments, different libraries may be mixed as they are introduced to a flow cell, and the identity of each sample may be determined as part of the sequencing process.

A subset of the single-stranded DNA library is then selected based on the size of the cfDNA to provide size-selected cfDNA library, wherein the size-selected cfDNA library has a DNA fragment length of 160 nucleotides or less, preferably 150 nucleotides or less, more preferably 140 nucleotides or less, even more preferably less than 115 nucleotides, and most preferably 110 nucleotides or less. Methods of size-selecting DNA fragments are well-known in the art and include the Pippin Prep system in which agarose gel electrophoresis is used to select a specified size of DNA by comparison to known DNA markers. Additional size-selection methods include enzymatic and bead-based methods. The size-selected cfDNA library is enriched for the ratio of mDNA to host cfDNA.

In an aspect, the ratio of mDNA to host cfDNA in the size-selected cfDNA library is enriched more than 50-fold, 75-fold, 100-fold, 150-fold, 200-fold or more compared to the total cfDNA. As shown in the Example, for an average sample analyzed using 11 million sequencing reads per library, this enrichment approach increases the total microbial DNA reads from 300 (using conventional double-stranded libraries) reads to 54,000 reads using size-selected single-stranded DNA libraries). This improved sensitivity makes the method described herein feasible for clinical use.

After the size-selected cfDNA library is prepared, the method may further comprise performing metagenomic sequencing on the size-selected cfDNA library. As used herein, metagenomic sequencing is sequencing all nucleic acids in a sample, which can contain mixed populations of organisms, including microorganisms. Next-generation sequencing allows the sequencing of tens to hundreds to thousands of organisms in parallel. Advantageously NGS metagenomic sequencing can provide detection of low-abundance microbes in a sample. The Illumina®-compatible adapters used in the SRSLY method allow for NGS metagenomic sequencing.

To identify the microbes represented in the ssDNA library, complete and plasmid genomes from human, bacteria and viruses can be downloaded from NCBI GenBank. For each candidate organism, reads classified to the corresponding species can be identified and expressed as a fraction of total sequencing reads that pass quality filters.

Exemplary sepsis-causing bacteria include Escherichia coli, Acinetobacter baumanii, Enterobacter sp., Bacteroides fragilis, Salmonella enterica, Shigella dysenteriae, Pseudomonas aeruginosa, Proteus mirabilis, Serratia marcescens, Neisseria meningitides, Klebsiella pneumonia, Streptococcus pneumonia, Streptococcus pyogenes, Streptococcus agalactiae, Staphylococcus haemolyticus, Staphylococcus aureus, Enterococcus sp. and combinations thereof.

Exemplary viral infections which can cause sepsis include herpes simplex virus, influenza, rhinovirus, parainfluenza virus types 1-3, respiratory syncytial virus, adenovirus, influenza B virus and coronavirus.

Exemplary fungal infections which can cause sepsis include Candida albicans and other Candida spp, Aspergillus spp., Histoplasma, and Pneumocystis jirovecci.

In an aspect, the methods described herein provide for personalized treatment of infections causing sepsis. In addition, the methods allow one to distinguish between true pathogens, background flora, and contaminants. And finally, the methods allow for faster turn-around time for the pathogen causing the infection compared to gold-standard testing using conventional microbiology.

In a further aspect, once a microbial infection has been identified, a therapy to treat the infection may be administered to the patient. Exemplary treatments for bacterial septic infections include treatment with broad spectrum antibiotics such as piperacillin/tazobactam, ceftriaxone, cefepime, meropenem, vancomycin, levofloxacin, ciprofloxacin, ceftazidime, and imipenem/cilastatin; administration of intravenous fluids; oxygen therapy; and/or administration of vasopressors to narrow blood vessels and increase blood pressure. Exemplary treatments for fungal septic infections include cchinocandins, triazoles, and amphotericin B. Exemplary treatments for viral sepsis include baloxavir, oseltamivir, peramivir and zanamivir for influenza infections and cidofovir for adenovirus infections in immunocompromised patients. The broad-spectrum antiviral drug ribavirin may be used for the treatment of patients with rhinovirus and respiratory syncytial virus infections, and arbidol for rhinovirus, respiratory syncytial virus, adenovirus and parainfluenza virus infections. Remdesivir and lopinavir/ritonavir may be used in patients with SARS-COV-2 infection.

In patients with infection relating to organ transplant, exemplary treatments include rifampicin, macrolides, fluoroquinolone, amoxicillin-clavulanate, cephalosporins, azole antifungals and acyclovir or valganciclovir for treatment of viral infections.

In patients with an altered gastrointestinal microbiome, treatment includes microbial replacement therapy or fecal microbial transplantation. Other treatments include probiotics, prebiotics, postbiotics, antibiotics, and the like.

In an aspect, non-native DNA includes donor derived cell-free DNA from transplanted organs as well as ctDNA.

In another aspect, a method of enriching a plasma sample from a host subject for ratio of circulating tumor DNA (ctDNA) to host subject cell-free cfDNA comprises obtaining the plasma sample from the host subject; extracting total cfDNA from the plasma sample; selecting a subset of the total cfDNA based on the size of DNA fragments in the cfDNA to provide size-selected cfDNA, wherein the size-selected cfDNA has a DNA fragment length of 160 nucleotides or less; and preparing a single-stranded DNA library from the size-selected DNA, wherein the single-stranded DNA library is enriched for the ratio of ctDNA to host subject cfDNA compared to the total cfDNA.

In patients with cancer, a variable fraction of cfDNA in plasma is contributed by cancer cells. These DNA fragments, known as circulating tumor DNA (ctDNA), carry tumor-specific somatic genetic alterations. Analysis of circulating cfDNA from plasma has several potential diagnostic applications in cancer medicine. For example, ctDNA can be a biomarker for pre-clinical cancer and post-diagnosis monitoring both during and after treatment. Without limitation, ctDNA can be used to identify genes altered in cancers and can identify single-nucleotide variants (SNVs), insertions/deletions (indels), fusions, and copy number alterations (CNAs). Exemplary ctDNA markers include:

| Cancer type | Markers |
|---|---|
| Prostate cancer | TP53, AR, HRR |
| Breast cancer | ESR1, PIK3CA, TP53, MUC1 |
| Ovarian cancer | KRAS, PIK3CA, BRAF, ERBB2 |
| NSCLC | KRAS, LKB1, ROS1, MET, EGFR, ALK, BRAF, |
| | NRAS, TERT |
| Pancreatic cancer | BRCA2, K-ras, KDR, EGFR, ERBR2 |
| Colorectal cancer | RAS, BRAF, APC, mSEPT9 |
| Gastric cancer | TP53, PIK3CA, FBXW7 |
| Melanoma | BRAF, NRAS, TERT |

In the treatment of patients with cancer, identification of ctDNA markers may be used to guide the course of chemotherapy.

Advantageously, the high resolution and low cost of the methods described herein will allow better characterization of microbial DNA from plasma samples, and development of microbial detection as a biomarker for diagnosis of sepsis and identification of pathogens in patients with cancer, transplant patients and patients with other infections.

The methods described herein may be used to create libraries in samples from mammals and non-mammals, particularly mammals.

The invention is further illustrated by the following non-limiting examples.

### Examples

### Methods

Study Cohort and Sample Collection: Patients were included from an on-going study (January 2022 till date) recruiting trauma patients admitted to the Intensive Care Unit (ICU) directly from the Emergency Department at University of Wisconsin Hospitals and Clinics. Serial blood samples were collected on days 1-10 of hospital admission. The first sample was collected within 24 hours of admission. For this analysis, a subset of patients with culture-proven infection was identified. The institutional review board of the University of Wisconsin-Madison approved this study (IRB protocol number 2021-0484). Study patients or their legal designees provided written informed consent to participate.

Blood Processing and Cell-Free DNA Extraction: Laboratory protocols optimized for cell-free DNA analysis for collection and processing of samples were used. At each time point, one to two 10 ml Streck Cell-Free DNA BCT® blood tubes were collected. Blood samples were processed within 24 hours of collection with two rounds of centrifugation to isolate plasma (820 g for 10 min; 20,000 g for 10 min) which was stored at −80° C. in 1-ml aliquots until further analysis. DNA was extracted using ThermoFisher MagMAX™ Cell-Free DNA extraction kit.

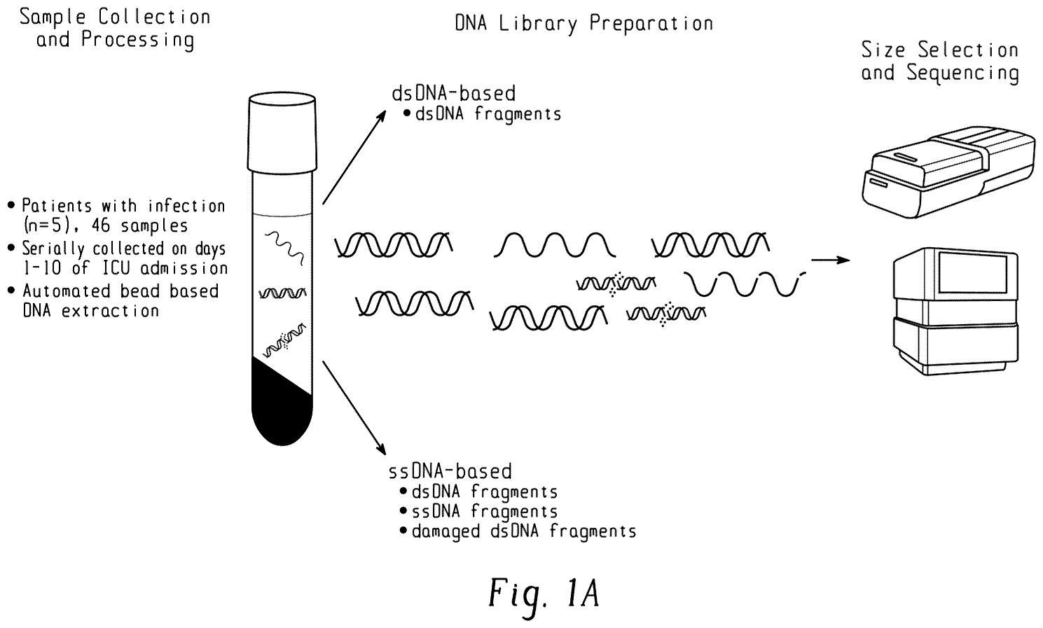

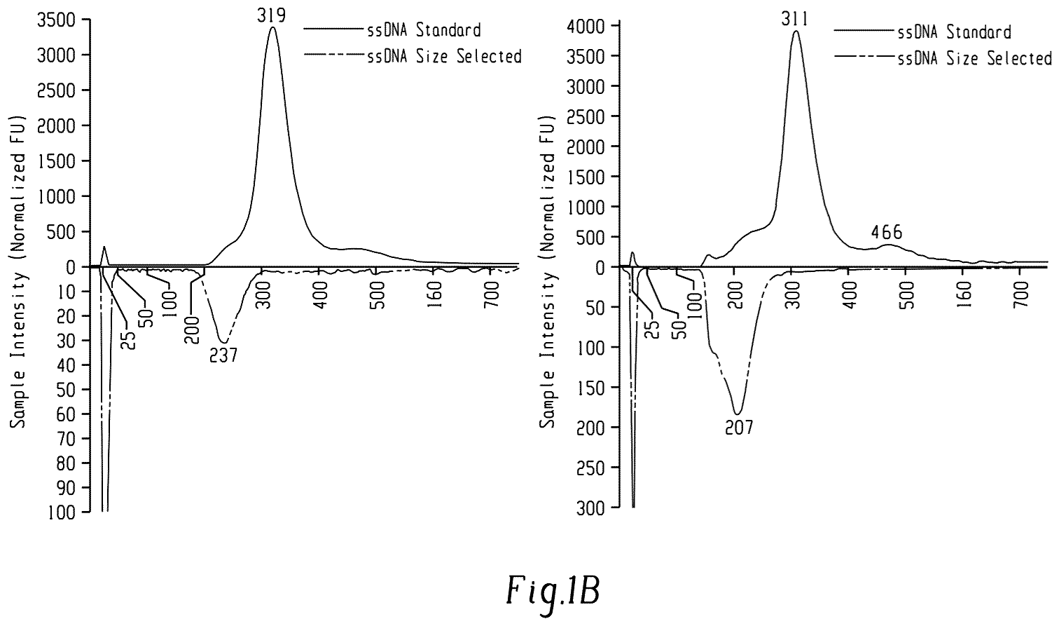

Library Preparation Assays and Sequencing: Matched libraries for sequencing were prepared from the same plasma DNA aliquot using a double-stranded (ThruPLEX® Plasma-seq kit from Takara Bio) and a single-stranded (SRSLY™ NGS Library Prep Kit from Claret Bioscience) DNA kit (FIG. 1). Sequencing libraries were pooled and sequenced on the Illumina® NextSeq 2000, generating a median of 11.4 million paired-end reads (2×50 bp) per sample.

Size-selection of mDNA fragments: Based on published results, human cell-free DNA fragments have a modal peak size of 167 bp while microbial DNA tends to be shorter in size due to a lack of histone-mediated protection from degradation. To enrich for mDNA, size selection was performed on pooled libraries using automated agarose gel electrophoresis (BluePippin™ from Sage Science) to exclude DNA fragments greater than 110 bp in length (FIG. 1A).

Analysis of microbial and pathogen-specific DNA: Sequencing reads were aligned to hgT2T v2.0 using bwa v0.7.17 (19) and samtools v1.15.1 to filter human reads out of the dataset. Read pairs in which at least one read was not mapped to the human genome were then extracted using samtools (samtools view-ubhF 3840-rf 12) for taxonomic classification. These reads were further filtered for quality using fastp 0.20.1 (fastp-pcl 25). Each fragment was categorized using the kmer-based taxonomic classifier Kraken 2 v2.1.2 with the prebuilt Refseq Kraken2 database (downloaded Jun. 7, 2022) and the taxonomic counts were refined at both the genus and phylum level using Braken 2.7. Microbial fractions were calculated as the ratio of fragments assigned to any microbial phyla over the sum of all fragments that were not filtered out by fastp (fragments assigned to microbial phyla/all fragments). For each microbe found in clinical cultures from a patient, the fraction of fragments assigned to the genus of that microbe was calculated for each sample from that patient. A negative correlation was observed between microbial fraction and total cell-free DNA concentration across all four library preparations. Thus, pathogen-specific DNA is reported as a ratio of fragments assigned to a known genus over microbial fragments (as opposed to all cell free DNA fragments).

Statistical Analysis: Comparisons of microbial DNA fractions between four library preparation conditions were performed using a Mann-Whitney U test. The correlation between microbial DNA fraction and cell-free DNA was assessed using a Pearson's correlation test with both variables log transformed. For each individual candidate genus (identified in microbiology culture), the fraction of mDNA derived from that genus across all samples was calculated. For the candidate genus, the number of median absolute deviations (MADs) that the measured genus-level mDNA fraction was away from the median across the set of samples from patients in whom that pathogen was not cultured was calculated. A potential pathogen would be considered significantly detected above background noise if the observed genus-level mDNA fraction in the sample was at least 2.5 MADs above the median. Two-sided p values are reported unless indicated otherwise. Data analyses were performed in Julia, with statistical analyses performed using the Statistics.jl and HypothesisTests.jl packages and plots were generated using Makic.jl.

### Results

Study participants and samples: 46 plasma samples obtained from 5 critically ill trauma patients who developed culture-proven infections during this study were analyzed. Overall, 17 of 46 samples (36.9%) were coincident with eight positive cultures observed (obtained day of positive culture of within 1 calendar day before or after).

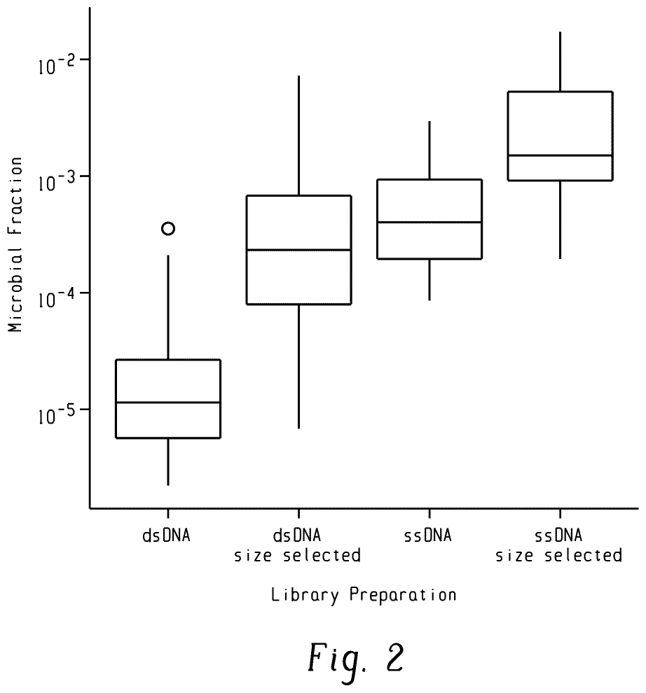

Type of library preparation and mDNA enrichment: Whole genome sequencing of all plasma DNA libraries was performed, generating a median depth of 11.4 million reads per library. The proportion of reads that did not map to the human genome increased from a mean of 1.2×10⁻³ in dsDNA libraries to 7.7×10⁻³ in the ssDNA libraries, resulting in a mean unmapped read enrichment of 5.4-fold (stdev 3.8, p<0.0001) between paired libraries. Using Kraken2 and Bracken to perform taxonomic classification, a mean proportion of 3.2×10⁻⁵ (stdev 6.1×10⁻⁵) of all sequenced fragments was classified as microbial at the phylum level using the dsDNA libraries (FIG. 2). This increased to 8.5×10⁻⁴ (stdev 7.5×104) using the ssDNA library preparation, with a mean fold increase of 43.1 (stdev 23.8, p<0.0001) between paired libraries.

Size Selection and mDNA enrichment: A method that leverages size differences between human and microbial cfDNA to enrich for mDNA was previously developed. Here, fragments shorter than 110 bp in length after library preparation and pooling were selected (FIG. 1B). Size selection significantly increased the proportion of microbial reads for both library preparations: from a mean of 3.2×10⁻⁵ to 7.4×10⁻⁴ (23-fold, p<0.0001) in dsDNA libraries and 8.5×10⁻⁴ to 4.9×10⁻³ in ssDNA libraries (6-fold, p<0.0001). This corresponded to an increase in reads that did not map to the human genome, from a mean of 1.2×10⁻³ to 3.6×10⁻³ in dsDNA libraries and 7.7×10⁻³ to 3.1×10⁻² in ssDNA libraries. Overall, using size selected ssDNA libraries instead of standard dsDNA libraries increased the mDNA fraction by a mean of 234.6 fold (stdev 163.2, p<0.0001) in libraries from paired plasma DNA samples (FIG. 2).

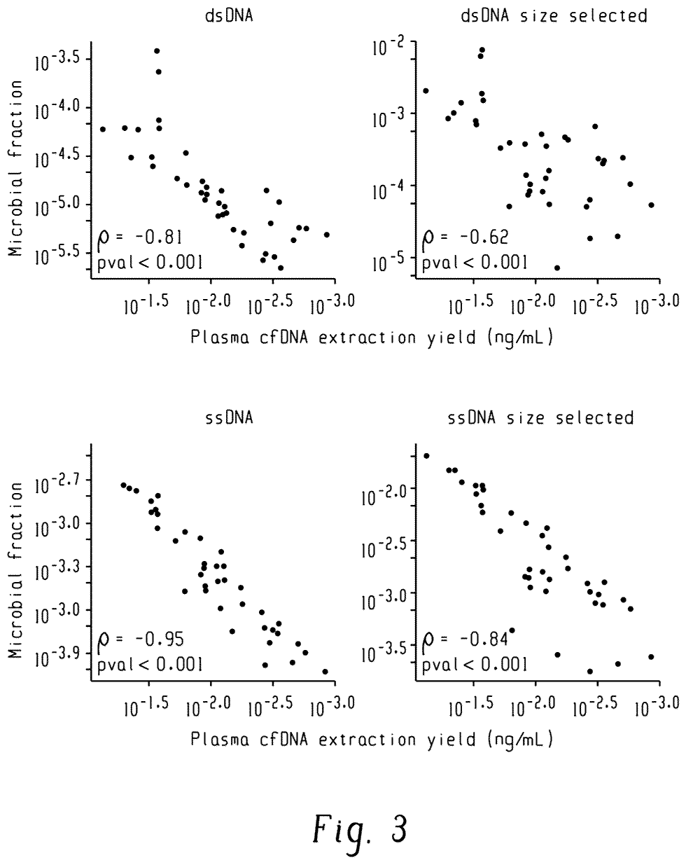

Microbial fraction and cell-free DNA: One key determinant of microbial DNA fraction was the amount of cfDNA extracted from one mL of plasma (FIG. 3). In this group of patients, the extraction yield was noted to be higher than yields commonly reported from healthy individuals and varied widely across samples, from 13.5 ng/ml to 836.0 ng/ml (median 89.4 ng/mL, stdev 172.7 ng/ml). The microbial DNA fraction was negatively correlated with extraction yield across all preparation types, with a correlation coefficient ranging from 0.64 for size-selected dsDNA libraries to −0.95 for ssDNA libraries.

Pathogen detection: A total of 33 microbiology cultures were obtained in the first 10 days of hospital admission across all five patients (blood, urine, and bronchoalveolar lavage/tracheal aspirate/sputum). Of these, eight cultures were positive with an identified pathogen (Staphylococcus aureus=5, Streptococcus pneumoniae=1, Klebsiella (Enterobacter) aerogenes=1, Haemophilus influenzae=2, Serratia marcescens/ureilytica=1) (Table 1) and treated with antimicrobials per physician discretion (Table 2). In total, 17 of the 46 plasma samples were taken within one day of a positive culture. The genera found in these positive cultures were compared with pathogen genus-specific mDNA fractions in the coincident plasma samples. To account for the influence of cfDNA extraction yield on the proportion of microbial DNA, the fraction of genus-specific reads over total microbial reads at each timepoint was calculated. At least one pathogen was detected significantly above background in seven, fourteen, twelve, and six of the 17 samples for dsDNA, size-selected dsDNA, ssDNA and size-selected ssDNA library preparations respectively (Table 3). Background noise associated with these genera in unrelated samples without positive cultures was highest in size-selected ssDNA and lowest in dsDNA libraries (Table 4). These results suggest that while microbial fraction is highest with the combination of ssDNA library preparation and size selection, this enrichment does not improve sensitivity for known pathogens in these samples. The preparation that provided the best tradeoff between pathogen sensitivity and sequencing requirements across samples was the size-selected dsDNA preparation.

TABLE 1
List of all microbiology culture results
for first 10 days of hospital admission.

| | Hospital | | Culture | |
|---|---|---|---|---|
| Patient | Day | Sample | Site | Microbiology Culture Results |
| B251 | 2 | T0 | Urine | No growth |
| B251 | 3 | None | Blood | No growth |
| B251 | 3 | None | Blood | No growth |
| B251 | 3 | None | Tracheal | Moderate Staphylococcus |
| | | | | aureus |
| | | | Aspirate | Moderate Streptococcus |
| | | | | pneumoniae |
| B251 | 4 | T2 | Urine | No growth |
| B251 | 9 | T7 | Blood | No growth |
| B251 | 9 | T7 | Blood | No growth |
| B251 | 9 | T7 | BAL | No growth |
| B251 | 9 | T7 | Urine | No growth |
| B251 | 10 | T8 | BAL | 5 × 10\hat{ }3 CFU/mL |
| | | | | Staphylococcus aureus |
| | | | | 1 × 10\hat{ }6 CFU/mL |
| | | | | Gardnerella vaginalis |
| B266 | 2 | T0 | BAL | 4 × 10\hat{ }5 CFU/ml |
| | | | | Staphylococcus aureus |
| B266 | 2 | T0 | Blood | No growth |
| B266 | 2 | T0 | Blood | No growth |
| B266 | 6 | T4 | BAL | No growth at 10\hat{ }3 CFU/mL. |
| B268 | 1 | T0 | Urine | No growth |
| B268 | 5 | T4 | Blood | No growth |
| B268 | 5 | T4 | Blood | No growth |
| B268 | 6 | T5 | BAL | 1 × 10\hat{ }5 CFU/mL Klebsiella |
| | | | | (Enterobacter) aerogenes |
| B268 | 6 | T5 | Urine | No growth |
| B268 | 10 | T9 | Blood | No growth |
| B268 | 10 | T9 | Blood | No growth |
| B297 | 6 | T5 | Urine | No growth |
| B297 | 6 | T5 | Blood | No growth |
| B297 | 6 | T5 | Blood | No growth |
| B297 | 6 | T5 | Sputum | Many Haemophilus influenza, |
| | | | | few Serratia marcescens/ |
| | | | | ureilytica |
| B297 | 7 | T6 | BAL | 1 × 10\hat{ }3 CFU/mL Haemophilus |
| | | | | influenzae |
| B297 | 7 | T6 | Blood | No growth |
| B297 | 8 | T7 | Urine | No growth |
| B304 | 2 | T0 | Urine | No growth |
| B304 | 2 | T0 | Blood | No growth |
| B304 | 2 | T0 | Blood | No growth |
| B304 | 2 | T0 | Sputum | Moderate to many |
| | | | | Staphylococcus aureus |
| B304 | 3 | T1 | BAL | 6 × 10\hat{ }3 CFU/mL |
| | | | | Staphylococcus aureus |

TABLE 2
List of antimicrobial treatment for the
first 10 days of hospital admission.

| Patient | Course | Hospital Days | Antimicrobials |
|---|---|---|---|
| 251 | 1 | 4-5 | Vancomycin |
| | 2 | 4-6 | Zosyn ® |
| | 3 | 5-10 | Ceftriaxone |
| 266 | 1 | 2-3 | Ceftriaxone |
| | 2 | 3-9 | Cefazolin and |
| | | | vancomycin |
| | 3 | 6-7 | Zosyn ® |
| 268 | 1 | 1-2 | Vancomycin |
| | 2 | 1-4, 6-10 | Zosyn ® |
| 297 | 1 | 1 | Cefazolin |
| | 2 | 6-8 | Vancomycin |
| | 3 | 7-8 | Zosyn ® |
| | 4 | 8-10 | Cefepime |
| 304 | 1 | 2-3 | Vancomycin |
| | 2 | 3-7 | Zosyn ® |
| | 3 | 7-10 | Augmentin ® |

TABLE 3
Detection of pathogens in paired samples across 4 library preparation
methods in samples coincident with positive cultures.

| | | | | Microbial Abundance (Reads per Million) | | | |
|---|---|---|---|---|---|---|---|
| | Hospital | | Pathogen | | dsDNA- | | ssDNA- |
| Patient | Day | Sample | genus | dsDNA | SS | ssDNA | SS |
| B251 | 2 | T0 | Staphylococcus | — | 1.7 | 0.61 | NS |
| | | | Streptococcus | 8.41 | 76.6 | NS | NS |
| B251 | 4 | T2 | Staphylococcus | — | 0.50 | — | NS |
| | | | Streptococcus | 5.66 | 22 | 5.5 | 1.76 |
| B251 | 9 | T7 | Staphylococcus | — | — | 0.70 | 9 |
| | | | Gardnerella | 0.25 | 0.37 | — | NS |
| B251 | 10 | T8 | Staphylococcus | — | 0.44 | 0.24 | NS |
| | | | Gardnerella | — | 0.44 | — | 0.259 |
| B251 | 11 | T9 | Staphylococcus | — | 0.53 | 0.39 | NS |
| | | | Gardnerella | 0.67 | 0.98 | 0.79 | 0.496 |
| B266 | 2 | T0 | Staphylococcus | 1.8 | 7.13 | 0.82 | NS |
| B266 | 3 | T1 | Staphylococcus | — | — | 1.85 | NS |
| B268 | 5 | T4 | Klebsiella | — | — | NS | NS |
| B268 | 6 | T5 | Klebsiella | — | 0.16 | 0.49 | NS |
| B268 | 7 | T6 | Klebsiella | — | 2.81 | 0.69 | 3.51 |
| B297 | 5 | T4 | Haemophilus | — | — | — | — |
| | | | Serratia | — | 2.81 | — | NS |
| B297 | 6 | T5 | Haemophilus | — | — | — | — |
| | | | Serratia | — | 0.54 | — | NS |
| B297 | 7 | T6 | Haemophilus | — | — | — | — |
| | | | Serratia | — | 4.16 | NS | — |
| B297 | 8 | T7 | Haemophilus | — | 1.02 | — | — |
| | | | Serratia | — | NS | — | 1.58 |
| B304 | 2 | T0 | Staphylococcus | 0.65 | — | 3.62 | NS |
| B304 | 3 | T1 | Staphylococcus | — | 5.95 | 3.53 | NS |
| B304 | 4 | T2 | Staphylococcus | 0.35 | 2.41 | 1.4 | NS |

There were 8 culture-proven infections in 5 patients. This table presents genus-specific mDNA fractions with 17 coincident plasma samples across the four library preparation methods where MAD scores were significant (>2.5 MADs above median).
NS, not significant and —, not detectable.

TABLE 4
Median background proportion for pathogen genera across samples
from patients where the given pathogen did not grow in culture.

| | Median background genus fraction | | | |
|---|---|---|---|---|
| Genus | dsDNA | dsDNA-SS | ssDNA | ssDNA-SS |
| Gardnerella | 0 | 0 | 0 | 0 |
| Haemophilus | 0 | 0 | 0 | 1.71E−04 |
| Klebsiella | 0 | 0 | 1.29E−04 | 6.99E−04 |
| Serratia | 0 | 2.07E−03 | 6.61E−04 | 6.41E−04 |
| Staphylococcus | 0 | 0 | 0 | 6.75E−04 |
| Streptococcus | 0 | 2.48E−03 | 7.69E−04 | 1.33E−03 |

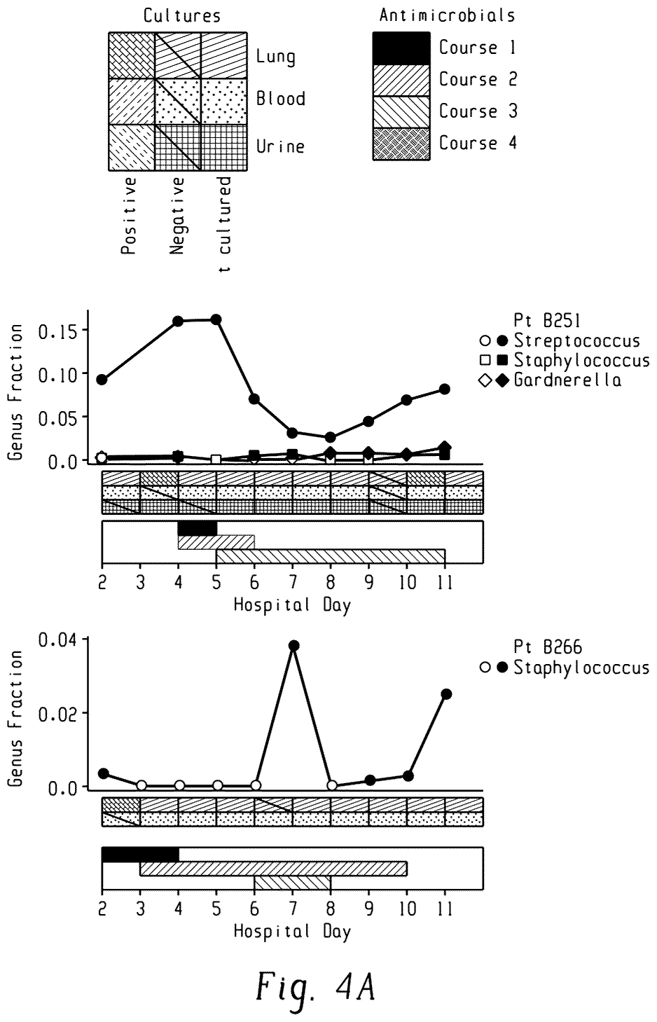

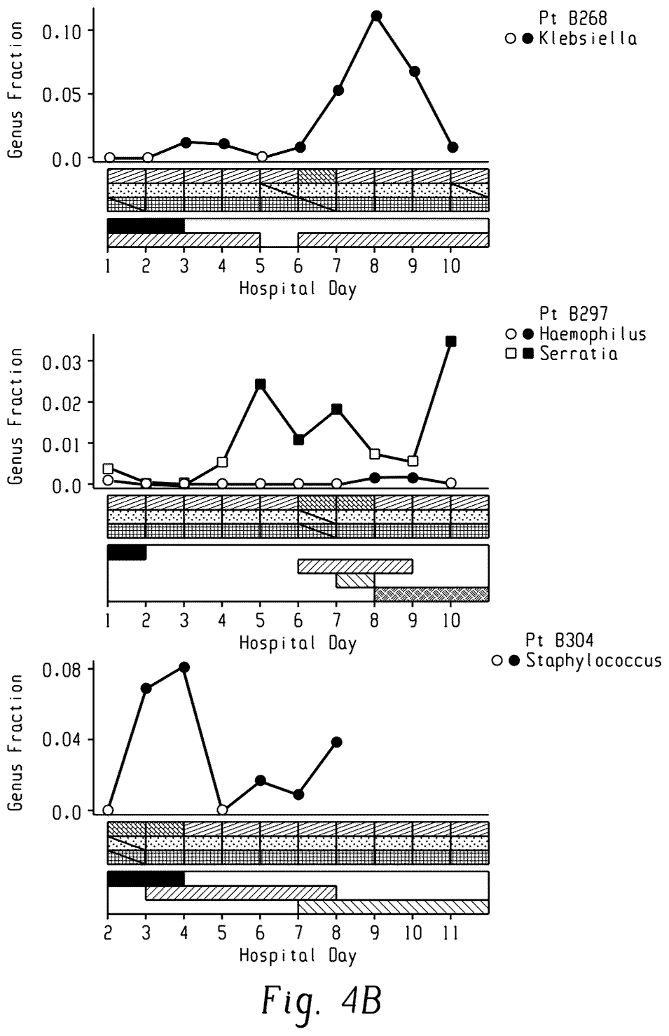

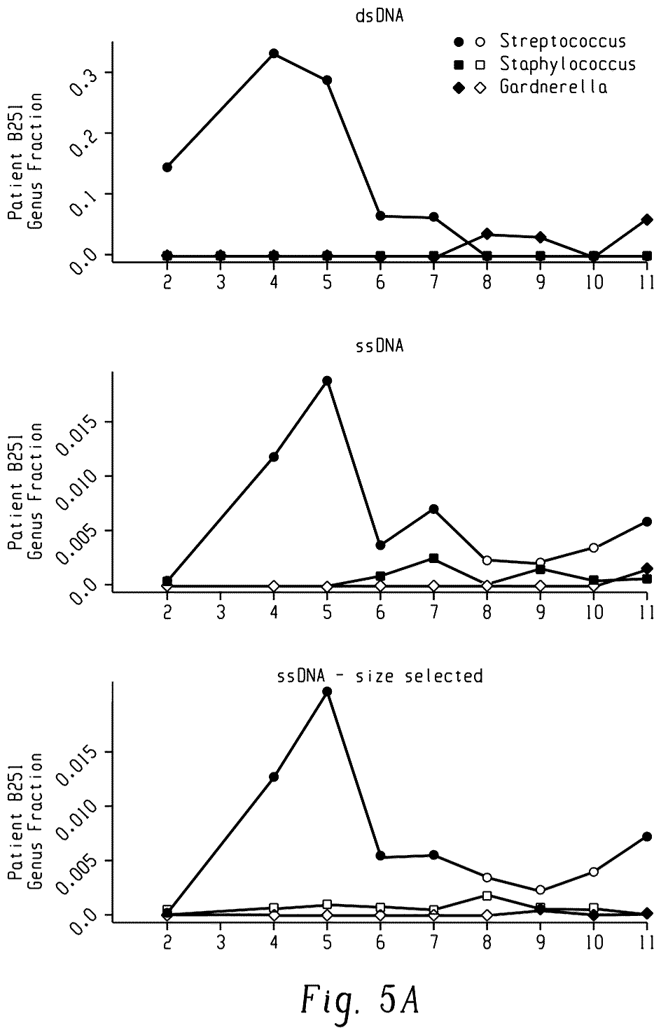

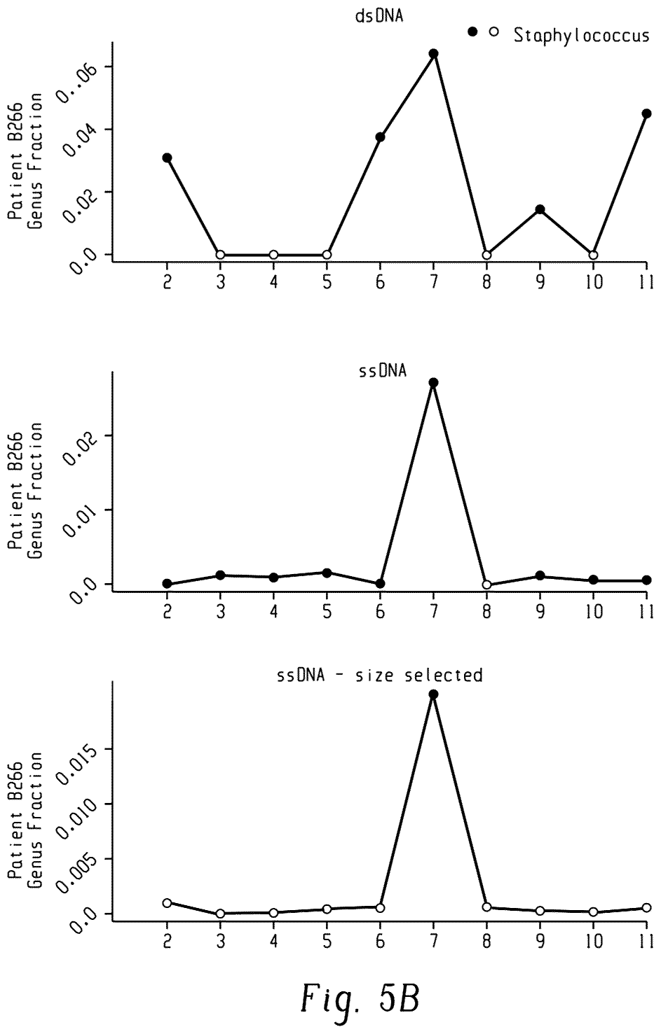

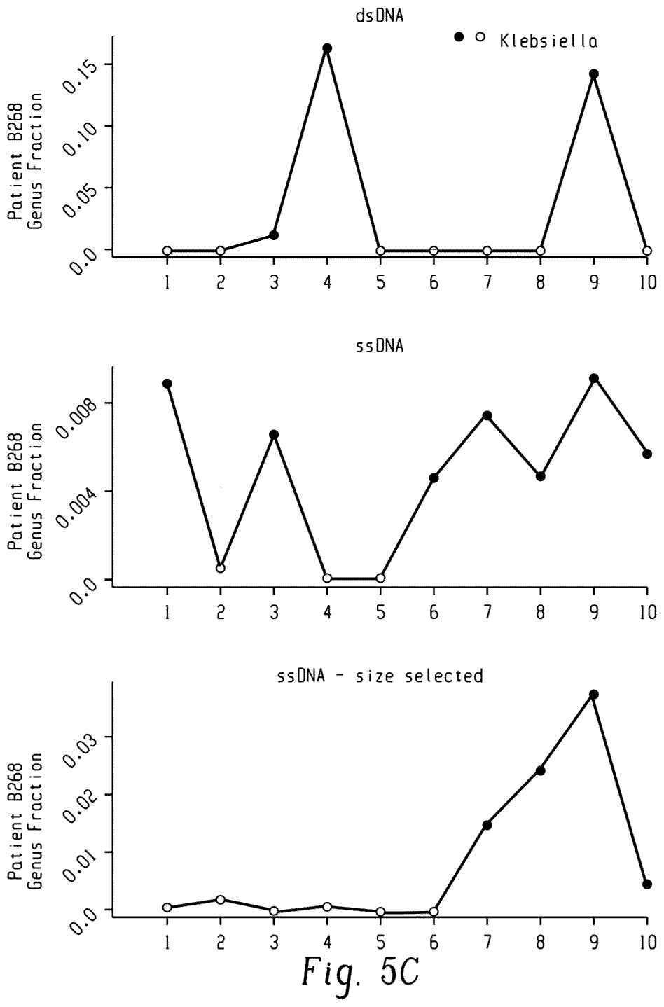

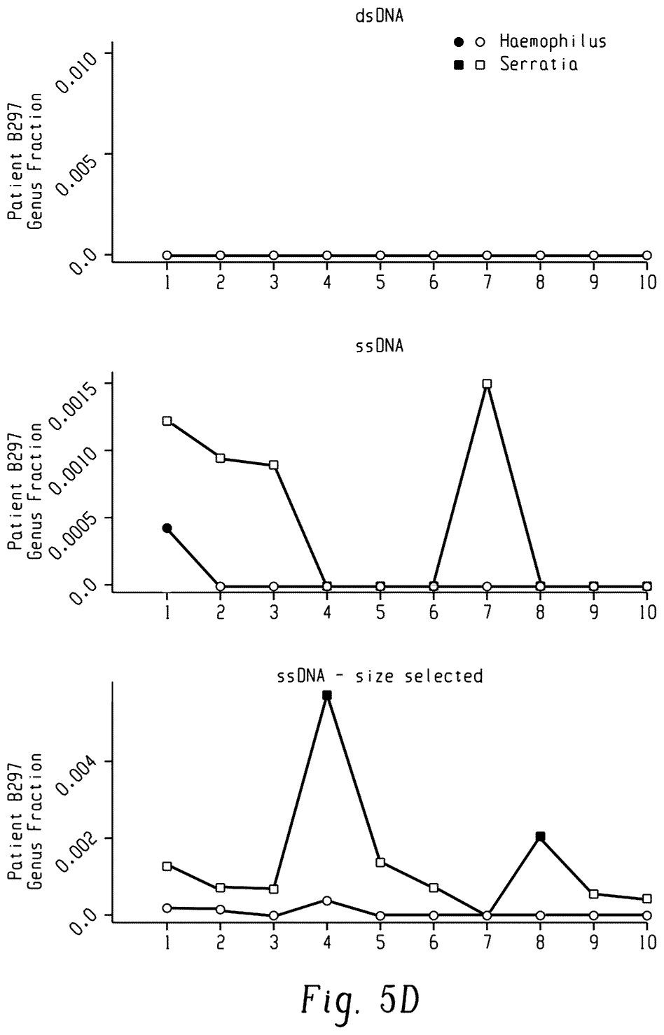

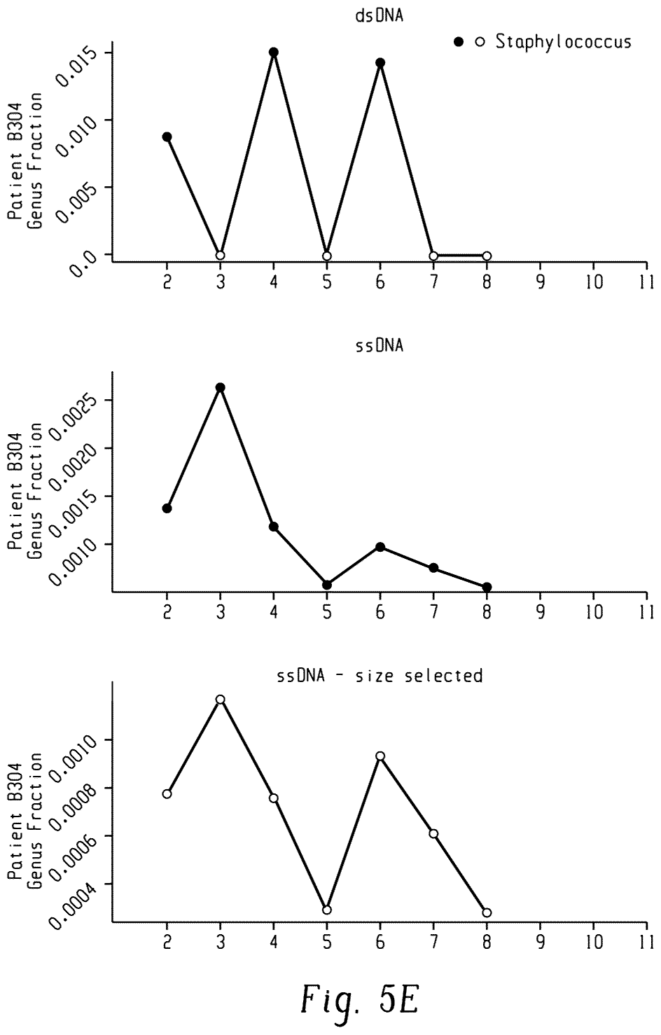

Pathogen detection and changes in pathogen fraction in longitudinal samples: The pathogen genus-specific DNA fraction was analyzed across all timepoints in each patient using size-selected dsDNA sequencing (FIG. 4) as well as the other three approaches (FIG. 5). In one example, patient B251 was diagnosed with polymicrobial pneumonia and had 2 positive respiratory cultures (on hospital day 3 and day 10). On hospital day 3, cultures grew Staphylococcus and Streptococcus. In plasma, DNA fragments from Staphylococcus and Streptococcus were detected on hospital day 2 and day 4 (no research sample was obtained on day 3). On hospital day 10, cultures grew Staphylococcus and Gardnerella. In plasma, DNA fragments from Gardnerella were detected on days 9, 10 and 11, as well as from Staphylococcus on days 10 and 11. For both episodes, pathogen-specific DNA was observed a day before a positive microbial culture was obtained. In addition, pathogen DNA levels for Streptococcus remained detectable above background throughout the sampling period, decreasing from days 5-7 as antimicrobial treatment was initiated but eventually increasing steadily to pre-treatment levels from days 8-11 Table 2). Another patient (B266) had 2 positive respiratory cultures (hospital day 2 and day 14), both growing Staphylococcus. In plasma, we detected DNA fragments from Staphylococcus on day 2. Although this became undetectable from days 3-6, we observed an increase in pathogen fraction on day 7, coinciding with a new respiratory culture obtained on day 6 that showed no growth. The patient showed persistent detection of Staphylococcus on days 9-11 and eventually another culture obtained on day 14 was positive for Staphylococcus (no additional research samples were available beyond day 11).

### DISCUSSION OF RESULTS

Recent studies have shown the relevance of plasma metagenomic sequencing to address the need for earlier diagnosis of sepsis in critically-ill patients. While promising, an outstanding challenge for this approach is the very limited concentration of microbial DNA in circulation, and the need for high depth of sequencing to detect microbial DNA fragments while filtering the more abundant human DNA fragments in circulation. Described herein is a paired comparison between four different approaches to enrich microbial DNA in plasma samples obtained from critically ill trauma patients with positive microbial cultures. As shown herein, microbial DNA can be enriched over 200-fold by using a combination of single-stranded DNA library preparation and size selection compared to conventional dsDNA sequencing. However, this does not translate into improved detection of pathogen-specific DNA above background noise. When we enriched dsDNA libraries for shorter DNA fragments, sensitivity for pathogen DNA improved from 41% (7/17 samples) to 82% (14/17 samples). In contrast, when ssDNA libraries were enriched for shorter DNA fragments, sensitivity decreased from 71% (12/17 samples) to 35% (6/17 samples).

In patients with sepsis, cell-free DNA concentration is variable and can range from a few nanograms to a few micrograms in each milliliter of plasma. Plasma DNA concentration ranged from 14 ng/mL to 836 ng/mL in critically ill patients with infection. Interestingly, the microbial DNA fraction was inversely correlated with total plasma DNA concentration, an observation that remained consistent across all four library preparations. Without being held to theory, it is hypothesized that this is due to increased human cfDNA in higher concentration samples, leading to a relative dilution of microbial DNA within host DNA. Since total cell-free DNA concentration is susceptible to multiple physiological as well as pre-analytical confounding factors, this observation implies comparisons in total and pathogen-specific microbial DNA fractions or pathogen detection between patients, or across longitudinal samples from the same patient should be adjusted for total plasma DNA concentration. For analysis of longitudinal changes in pathogen DNA fraction during ICU stay in thus study, this challenge was overcome by calculating pathogen fraction relative to the total reads classified to be of microbial origin, instead of total number of sequencing reads generated.

Despite the promise of mDNA analysis for detection of pathogens in sepsis, the very low abundance of mDNA in plasma (3.4 Reads Per Million in a cohort of patients with sepsis) renders detection difficult. Recent work has focused on how mDNA is different from human cell-free DNA (cfDNA) in plasma to enrich for mDNA. mDNA is shorter and more fragmented than human cfDNA. The standard double-stranded DNA (dsDNA) library preparation methods are more effective in capturing double-stranded fragments with blunt ends and overhangs. In comparison, single-stranded DNA (ssDNA) library preparations can not only capture double-stranded fragments but also single-stranded fragments and fragments with nicks. ssDNA library preparations were originally developed and used for the genomic analysis of highly degraded ancient DNA. Recently, they have been adopted for other fragmented sample types such as mitochondrial and microbial cfDNA. The results provided herein demonstrate a 43-fold increase in microbial DNA fraction in ssDNA library preparation over conventional library preparation in paired samples. This furthers the understanding of microbial DNA being highly degraded in plasma as compared to human cfDNA.

Previous studies have performed high-depth plasma sequencing to obtain adequate mDNA reads for analysis. For example, the Karius test which is a validated microbial DNA sequencing test uses on average 24 million reads per sample in their first study and >20 million reads per sample in subsequent studies for mDNA analysis (Blauwkamp T A, Thair S, Rosen M J, Blair L, Lindner M S, Vilfan I D, et al. Analytical and clinical validation of a microbial cell-free DNA sequencing test for infectious disease. Nat Microbiol 2019; 4:663-74). Another recent example of a mDNA sequencing test used on average 134 million reads per sample to ensure adequate microbial reads within majority human cfDNA reads (Wang G, Lam W K J, Ling L, Ma M L, Ramakrishnan S, Chan D C T, et al. Fragment ends of circulating microbial DNA as signatures for pathogen detection in sepsis. Clin Chem 2023; 69:189-201). The authors described that the detection rate of mDNA of a genus is mainly related to two factors in a plasma sample of a patient with sepsis, which is sequencing depth and abundance of the genus. They described an in-silico size selection for shorter cfDNA fragments and concluded that such a method can reduce sequencing needs from 57 million to 14 million total sequenced fragments, while still having sufficient reads for analysis. In the experiments described herein, it is demonstrated that selecting for fragments less than 110 bp before sequencing enriches mDNA fraction in both types of library preparation (23-fold for size-selected dsDNA libraries vs 6-fold for size-selected ssDNA libraries). To provide context, for an average sample in the study with 11 million reads per library (median depth in this study), total mDNA fragments detected increased from 300 (using conventional dsDNA libraries) to 8000, 9000 and 54,000 using size-selected dsDNA, ssDNA, and size-selected ssDNA libraries, respectively. In comparison, Wang et al. reports an average of 36.7 reads per million (microbial abundance) in patients with sepsis with an average sequencing depth of 134 million reads per sample. Thus, the approach described herein will make it possible for any future studies using microbial DNA as an analyte to obtain sufficient microbial DNA reads at lower sequencing depths. This also substantially lowers the cost of using a metagenomic sequencing based test for infectious disease diagnostics.

A metagenomic sequencing assay for detection of causative pathogen in sepsis should preferentially enrich for infection-derived mDNA amidst a background of commensal DNA, contaminating mDNA, and human cfDNA. It was observed that a size-selected ssDNA library preparation recovers over 200-fold more microbial DNA as compared to the conventional library preparation protocol. However, the library performance for pathogen detection is worse compared to ssDNA libraries or size-selected dsDNA libraries. Even though the mDNA from the pathogen known from microbiology cultures was detected in 94% of plasma samples (16/17) using size-selected ssDNA analysis, it is only significantly different from the background for the same pathogen in 35% of samples (6/17). Thus, it appears that the combined approach of single-stranded library preparation and size-selection enriches for both, infection-derived mDNA (signal) and contaminating mDNA (background noise) at the genus level. In contrast, the size-selected dsDNA protocol showed the best performance, detecting mDNA from the known pathogen in 82% of the samples. Considering the much higher number of microbial fragments obtained from size-selected ssDNA libraries, future studies could evaluate species-level classification or other informatic methods such as analysis of mDNA fragment ends to differentiate infection-derived mDNA from contaminating mDNA.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of enriching a plasma sample from a host subject for ratio of non-native cell-free DNA (cfDNA) to host subject cfDNA, comprising
obtaining the plasma sample from the host subject;
extracting total cfDNA from the plasma sample;
preparing a single-stranded DNA library from the total cfDNA, wherein the single-stranded DNA library is enriched for cfDNA existing in a single-stranded configuration; and
selecting a subset of the single-stranded DNA library based on the fragment size to provide a size-selected cfDNA library, wherein the size-selected cfDNA library has a DNA fragment length of 110 nucleotides or less and is enriched for the ratio of non-native cfDNA to host cfDNA.

2. The method of claim 1, wherein the non-native cell-free DNA (cfDNA) is microbial cell-free DNA (mDNA), and the size-selected cfDNA library enriched for the ratio of mDNA to host cfDNA.

3. The method of claim 1, wherein the non-native DNA comprises donor derived cell-free DNA from transplanted organs.

4. The method of claim 1, wherein the non-native DNA is circulating tumor DNA (ctDNA).

5. The method of claim 1, wherein the body fluid sample is a plasma sample, a urine sample, a sputum sample, a bronchoalveolar lavage, a stool sample, peritoneal fluid, pleural fluid, cerebrospinal fluid, synovial fluid, or interstitial fluid.

6. The method of claim 1, wherein the sample is a plasma sample.

7. The method of claim 1, wherein extracting total cfDNA comprises cell lysis followed by DNA isolation.

8. The method of claim 1, wherein the single-stranded DNA library is prepared by a method comprising a phosphorylation/ligation dual reaction with forward and reverse dsDNA next generation sequencing (NGS) adaptors containing single-stranded overhangs.

9. The method of claim 8, wherein, prior to the phosphorylation/ligation dual reaction, the DNA is coated with single-stranded DNA binding protein.

10. The method of claim 8, wherein, prior to the phosphorylation/ligation dual reaction, the DNA is heat denatured and cold shocked to produce single-stranded DNA, followed by coating the single-stranded DNA with single-stranded DNA binding protein.

11. The method of claim 8, wherein, prior to the phosphorylation/ligation dual reaction, the DNA is not heat denatured to produce native single-stranded DNA library.

12. The method of claim 1, wherein selecting the single-stranded DNA library based on the fragment size comprises agarose gel electrophoresis, enzymatic size selection, bead-based size selection, or a combination thereof.

13. The method of claim 12, wherein the ratio of non-native cfDNA to host cfDNA in the size-selected cfDNA library is enriched more than 50-fold compared to the total cfDNA.

14. The method of claim 1, further comprising performing metagenomic sequencing on the size-selected cfDNA library.

15. The method of claim 2, further comprising, based on the metagenomic sequencing, identifying one or more sepsis-causing pathogens.

16. The method of claim 15, further comprising administering a therapy to treat the one or more sepsis-causing pathogens.

17. The method of claim 2, wherein the plasma sample is from a blood sample drawn during hospital admission of the host subject.

18. The method of claim 2, wherein the plasma sample is from a blood sample drawn after organ transplant in the host subject.

19. The method of claim 4, wherein the ctDNA is a biomarker for pre-clinical cancer and post-diagnosis monitoring during or after treatment.
